(19) United States
(12) Patent Application Publication
(10) Pub. No.: US 2008/0138387 A1
(43) Pub. Date: Jun. 12, 2008

(54) Hemostatic sponge and article

(76) Inventor: Venkat R. Machiraju, Pittsburgh, PA (US)

Correspondence Address: Rama Nath, 2530, Quail Run, Lansdale, PA, 19446, US

(21) Appl. No.: 11/903,224

(22) Filed: Sep. 20, 2007

Related U.S. Application Data
(60) Provisional application No. 60/873,498, filed on Dec. 7, 2006.

(51) Int. Cl. A61L 15/32; A61L 15/54

(52) U.S. Cl. 424/446; 424/94.64; 602/44; 602/48

(57) ABSTRACT

A hemostatic article, e.g., a sponge is pretreated with predetermined quantities of selected hemostatic agents to assist hemostasis in surgical or traumatic wounds. The hemostatic article is provided with a visible marking/indication of the strength of the hemostatic agent. The visible indication may be color coding, or, the marking could take the form 20%, 40%, 60% and, 80%. The hemostatic agent may be any substance assisting in hemostasis, may be in powder or fluid form, and may be one or more of Thrombin, Gelform, Fibrinogen, or other hemostatic substances. The hemostatic article could include at least one radio-opaque element which may take the form of a thread or a strip. Alternatively, a radio chip embedded in the hemostatic article may be used. The visible markings per se could be made radio-opaque, thereby obviating a separate radio-opaque element. The pretreated marked hemostatic articles are sterilized for medical use in any known manner.

### RELATED APPLICATIONS

Benefit is claimed under 35 USC 119(e) to U.S. Provisional Application Ser. No. 60/873,498 entitled “Hemostatic Sponge and Article”, by Venkat R Machiraju, filed on Dec. 7, 2006, which is incorporated in its entirety herein by reference.

### FIELD OF THE INVENTION

This invention relates generally to fabric gauzes and sponges for medical use, and more particularly to hemostatic articles including gauzes and sponges which are meant be used during and in the context of surgery or for dressing bleeding wounds.

### BACKGROUND OF THE INVENTION

Hemorrhage of a blood vessel, body tissue or organ can result in blood loss leading to hypovolemic shock and death. However, despite continued advances in trauma care, a significant number of trauma victims suffer fatal or severe hemorrhage every year. Many of these fatalities could be prevented if adequate means existed for on site control of blood loss. Hemophiliacs and patients receiving anticoagulant medication (e.g., during and/or after heart surgery) are also at high risk for rapid blood loss. For such patients, recombinant antihemophilic factors have been known to be selectively used.

Gauzes: Surgical grade gauzes are used generally for the purpose of stopping excessive bleeding, and such gauzes are invariably made of fabric. Surgical grade gauzes exhibit high absorbency and are known to be used as swab or compress for cleaning, packing and dressing wounds.

More commonly, medical absorbent-gauzes are made of 100% woven cotton yarn. The cutting, folding and packing of the gauze is done in a way which ensures safety in its every possible use. Medical absorbent gauzes are manufactured by advanced technological machinery and later tested for quality assurance to meet quality and safety benchmarks while minimizing loose threads on the edges. They are available in various sizes, ply and thread density, in different sizes and even in jumbo cartons according to the customer request. Available medical absorbent gauze material generally is capable of withstanding steam sterilization at 134° C. and with Ethylene Oxide.

Sterilized absorbent gauzes are used as surgical sponges to facilitate the control of bleeding in outpatient procedures such as but not limited to loop electrosurgical excision procedure (LEEP), colposcopy, cryoablation, dilation and curettage (D & C) procedure, conization, and hysteroscopy. Surgical sponges can also be used in inpatient surgical procedures such as caesarian section, hysterectomy, fibroid surgery, laparoscopy, and oncological surgery. Some types of silky gauzes are easy to handle and can be customized in their configuration to fit into hard-to-reach bleeding sites that are often a challenge faced by obstetricians and gynecologists. During any type of surgery whether it is major or minor, bleeding is a problem. Bleeding during surgery is generally controlled by the use of Electrocautery, applying hemostat clamps and tying the knot with silk or nylon, or stitching it or applying gauze under pressure and holding the pressure for some time until the bleeding is controlled. Hemostat clamps are available in a wide variety of designs to suit specific surgical needs, and always have attendant disadvantages during use. Other known gadgets for hemostasis are argon-beam coagulator, microwave scalpel, and lasers.

It is known that to a significant extent, fabric gauzes/articles can be used in lieu of hemostat clamps and gadgets for controlling bleeding especially during surgery. It is further noted that a considerable amount of minor surgery is done in the offices by several non-surgeons as well. Surgeons and non-surgeons alike use dry gauze and hold the pressure to stop bleeding. Generally, bleeding stops in 3-5 minutes, and then the doctor applies gauze dressing and completes the procedure. If it is an outpatient procedure, the patient is sent home, or, if in the hospital, the patient is sent from the operating room to a recovery room. In some situations where patient's clotting mechanism is not effective or deficient, the patient might continue to bleed longer than usual. In other scenarios, a patient after an outpatient procedure might be sent home after bleeding has ceased. However, the wound might start bleeding again, either because of reasons such as an insufficient platelet count or because the patient caused the surgery area to be disturbed. The post operative patient would have to be brought back for a repeat visit so that the bleeding problem could be addressed.

Hemostatic agents and coagulating substances: To assist clotting, some selected topical agents are known to be used in medical practice. These agents are known in the art as hemostatic agents. Examples of hemostatic agents commonly available include Thrombin, Gelform and Cellulose, Aminocaproic acid, Tranexamic acid, Aprotinin®, Desmopressin® and ferric sulfate, to name a few. Another hemostatic agent which has been used with benefits in different types of surgery is Fibrin Sealant, known as FS. Another example of a commercial hemostatic agent is QuickClot® marketed by Z Media Corporation of Connecticut 06492, of the United States. Other hemostatic agents are known to those skilled in the art.

The hemostatic agents referred to supra, if in liquid form, are known to be supplied in syringes and are applied directly over the wound. However, the amount of the hemostatic agent applied is difficult to match with the patient requirement, and it is also difficult to make a repeat application of exactly the required strength of the hemostatic agent as necessary.

The use of fibrin as a coagulating substance for stopping bleeding and for sealing wounds has been widely accepted. Generally, such biological adhesives or “fibrin glues” are based on a two component system of fibrinogen and thrombin which when mixed form a fibrin coagulum by the cleavage of fibrinogen through the action of thrombin to form fibrin monomers that spontaneously polymerize to form a three dimensional network of fibrin. For example, Tisseel® is a two-component kit containing a fluid thrombin component including calcium chloride and a somewhat more viscous fibrinogen component including factor XIII, fibronectin, aprotinin and plasminogen. The two components are delivered deep frozen in two separate syringes, or as two lyophilized powders with corresponding aprotinin and calcium solutions as solvents. Using this method, the fibrin glue consolidates when the two components are combined due to fibrin monomer aggregation. The setting rate is dependent on the thrombin concentration and varies from a few seconds (high thrombin concentration) to a couple of minutes (low thrombin concentration).

The major disadvantage of these preparations is that the water-like fluidity of the components renders them difficult to handle and administer. Although various efforts have been made to facilitate the administration of these compositions, for example, by the development of double-syringe applicators as described in U.S. Pat. No. 4,359,049 or a spray system as described in U.S. Pat. No. 4,427,651, the basic problem of low viscosity has still posed problems.

U.S. Pat. No. 5,631,011 to Wadstrom describes a method of increasing the viscosity of fibrin glue compositions by adding a biocompatible polymer capable of forming a viscous aqueous solution. The components of this system are provided in deep freeze solutions or as lyophilized powders which are diluted prior to use with aqueous solutions. The primary route of administration taught by Wadstrom is by a two-component preparation. Thus, while Wadstrom emphasizes the advantages of increasing the viscosity of the solution over prior “water-like” fibrin glues, the components of this system still require additional preparation at the time of use. Wadstrom further teaches that compositions having a high viscosity are to be avoided because fibrin polymerization and adhesion to the tissues would be inhibited.

One currently used alternative to fibrin glue is a biodegradable collagen patch (“TAF” patch). For example, Zimmerman and Schiele, in U.S. Pat. No. 4,453,939, describe collagen carriers in the form of a foam, web or film that is coated with a mixture of blood-clotting components including fibrinogen and thrombin. To prevent reaction of the blood-clotting components prior to use, they are provided as a suspension in an organic solvent which is applied to the collagen by brushing, spraying or dipping. However, difficulties in achieving optimum timing for the fixing procedure have been reported using this method resulting in inconsistent attachment and stability of the active components onto the collagen carrier. Moreover, penetration of the active components beyond the surface of the collagen carrier is not possible using this method, thereby limiting the concentration of blood-clotting components available to the surface of the device. Another problem with the TAF patches is that the collagen fleece/foam used does not provide sufficient mechanical support once wet, preventing application of manual pressure to assist in stopping blood flow or repositioning of the patch once it has been applied to the wound. Further, surgeons have reported that the inflexibility of the TAF patch per se prevents them from easily conforming to the contours of the site to which they are applied. Still another problem with TAF patches is that they require refrigeration, prohibiting use outside a clinic or hospital setting.

The use of collagen for preparing a hemostatic article is taught in U.S. Pat. No. 4,404,970 to Sawyer. The Sawyer patent proposes to modify a sponge or a pad by including collagen or a collage-like substance, by using non-covalent modification of the collagen. In the Sawyer patent, in one form, collagen-like substance may be a gelatinous substance treated with hydrochloric acid. The Sawyer patent cross references U.S. Pat. No. 3,364,200 to Ashton, which teaches that oxidized cellulose has not only hemostatic properties but also was absorbable in animal tissue.

U.S. Pat. No. 3,364,200 to Ashton et al discloses surgical hemostats consisting of conventional gauze pads or similar articles impregnated with a hemostatic material such as ferric chloride, thrombin, or the like, which have been used for many years to arrest bleeding. It is noted that the focus and thrust in the Ashton patent are regarding how to produce an absorbable grade of oxidized cellulose, since removal of the hemostat from the bleeding site would disrupt any blood clot which has already formed, and might cause renewed bleeding. Ashton et al observed, therefore, that a vital need existed for a hemostatic material which could be left in place in a closed wound without causing serious local tissue reaction. It is also reported that previously, improvement was provided when it was discovered that oxidized cellulose not only had hemostatic properties but also was absorbable in animal tissue. Ashton et al provide oxidized cellulose absorbable hemostats having improved stability against deterioration on storage. The oxidized cellulose as taught in the Ashton patent is derived from wood pulp, cotton, cotton linters, ramie, jute, paper and similar materials and regenerated cellulose or rayon. However, notably, the prior art hemostats of the type exemplified by the Ashton patent are presently criticized in that they cannot be left in situ in a closed wound since an adverse reaction to foreign tissue would result, this being a serious disadvantage.

On the other hand, the present day trend is certainly not in favor of leaving hemostatic material in place after surgery, since expectations of re-absorption of the sponge material have resulted in problems. Further, there have been documented and publicized instances where surgery patients suffered significantly because of sponges left in cavities inadvertently, sometimes causing gossypiboma.

Publication WO 96/17633 describes tissue sealants including a fibrin bandage. In the method used for generating the bandage described in the WO publication, the active components are lyophilized in separate layers which are supported by an occlusive backing. Therefore, the active components are not homogeneously mixed throughout the bandage.

U.S. Pat. No. 6,056,970 to Greenwalt et al, dated May 2, 2000, teaches solid hemostatic compositions containing a bio-absorbable polymer and a hemostatic compound.

Radio-opaque elements in gauzes: In recent years, surgical sponges are available with embedded radio-opaque threads or strip which can easily be seen by an x-ray. Some hospitals routinely perform portable x-ray screening in the operating room before completing surgical procedures to ensure that no such sponges have inadvertently been left inside the patient. It is noted that without the radio-opaque inserts or markers, inadvertently retained sponges are difficult, if not impossible to diagnose. Accordingly, there is a need for improved hemostatic applicators or sponges that are sturdy enough to withstand manual pressure and which have a predetermined expected efficacy and convenience, especially during surgery, and in emergency situations such as life-threatening traumas wherein stemming blood flow as fast as possible can be critical.

### SUMMARY OF THE INVENTION

This invention teaches hemostatic articles pretreated with predetermined quantities of selected hemostatic agents for assisting hemostasis or otherwise stemming blood flow from surgical or traumatic wounds, where the hemostatic articles are provided thereon with a visible marking/indication of the hemostatic material strength/content. For purposes of this invention, the term “article” is to be understood to include sponge, gauze, bandage and articles of any configuration that are used in dressing and assisting hemostasis.

In one form, the invention resides in a hemostatic article which is pretreated with a predetermined quantity of one or more hemostatic/coagulating agents, the hemostatic article including a visible indication of the predetermined strength/quantity of the one or more hemostatic agents. The hemostatic agent may be in a powder form whereby when the pretreated article with dry powder upon use contacts blood, the chemical in the powder gets activated to promote blood clotting. Optionally, the present invention in one form includes the use of a radio-chip embedded into a sponge which transmits a signal to a receiver, for later diagnosis. However, these new sponges with embedded chips are relatively sophisticated and need external radio receiver-equipment for diagnostic purposes.

In a second form, the invention resides a hemostatic sponge including gauze material which is pretreated with a predetermined quantity of one or more hemostatic/coagulating agents, the hemostatic sponge including a visible indication of the predetermined quantity of the one or more hemostatic agents, the visible indication comprising markings on each hemostatic article to indicate a percentage content of the predetermined quantity of one or more hemostatic/coagulating agents.

The invention in another form resides in a hemostatic sponge including gauze material which is pretreated with a predetermined concentration of one or more hemostatic agents, the hemostatic sponge including a visible indication of said predetermined concentration of said one or more hemostatic agents, said visible indication comprising markings on each said hemostatic article to indicate a percentage content of said predetermined quantity of one or more hemostatic agents.

The present invention is more specifically directed to hemostatic sponges and gauzes which have been pretreated with a predetermined quantity of a selected hemostatic agent or agents, wherein the treated sponges are marked as to the percentage of the hemostatic agent applied, for easy identification during manufacture, shipping, storage, use and application. For purposes of this invention, the hemostatic agent or hemostatic agent/substance is intended to include one or more substances that would assist in causing hemostasis or otherwise assist stemming blood flow.

It is noted in this context that surgical gauze in one form is just cotton material and is available in packets of ten for instance. Every surgical procedure uses gauze sponges, either to wipe bleeding or to control bleeding by applying pressure. Gauze being a rough and foreign material, promotes clot formation. Second, it is malleable and conforming so it can be applied in any cavity in the body and in any corner. Additionally, sponges do not hurt the body tissues like metal hemostats or clamps do. Consequently, surgical gauze or sponges can be used freely, and all the sponges are removed at the end of the operation and are counted at the end.

With thromboplastic agents like Thrombin, Fibrinogen in a suitable form and Gelform when made as part of the gauze sponge itself in predetermined quantities, surgeons can use the hemostatic sponge instead of the regular sponge. It is proposed herein to spray or otherwise embed or apply chosen hemostatic agents in predetermined strengths such as for example: 20%, 40%, 60% or 80%, or any other preferred strengths on the sponges and identify the strength on the hemostatic agent applied thereon. Methods of application of a selected hemostatic agent, or combinations of hemostatic and other materials, into the treated sponges are well known to those skilled in the art. Hemostatic sponges thus pretreated, marked and prepared according to the invention can be distinguished as to their hemostatic strength/content and marketed at different price ranges. The marking of the treated sponges could take the form of imprinting the percentage strength, e.g., 20%, 40% or 60%, on the surface of the treated sponge (on one or both sides) or just underneath one fabric layer thereof. The percentage markings may be any other preferred figures, such as 25%, 50% and 75%, or even 1, 2, 3, 4, 5, as desired. After the marking is done, the treated sponge is subjected to sterilization as desired.

Advantageously, the imprinted characters representing the hemostatic content strength may be made radio opaque, thereby retaining the desirable feature of radio opacity in the treated sponge. Alternatively, the treated sponges may have printed characters thereon representing the hemostatic content, e.g., 20%, and the treated sponge could have an embedded distinct radio opaque thread or strip as desired. In a modified form of the invention, where it is preferred to have a distinct radio opaque thread or strip embedded in the treated sponge, the hemostatic content identification may be obtained by a predefined color coding of the sponge, the color coding being visibly displayed on the box of the sponges as well as the sponges themselves. Yet other methods of displaying the hemostatic strength/content of the treated sponge thereon may be resorted to, as desired by the users, and are within the ambit of the invention.

In a modification, the imprinted characters representing the hemostatic content strength may be displayed on both sides of a treated sponge, for easy visibility to the user, regardless of the orientation of the treated sponge in use. In a further modification, the imprinted characters representing the hemostatic content strength can be duplicated on each side of the treated sponge so as to be visible in multiple locations of the same side of the treated sponge for ready recognition. In such case, the imprinted characters may have to be reduced in size. If the imprinted characters can be made radio-opaque, it would diminish the difficulty for diagnosing any gossypiboma. Gossypiboma as known in medical practice is the pseudotumour within the body that is composed of non-absorbable surgical material with a cotton matrix. The most common cause of gossypiboma is an inadvertently retained surgical sponge. Surgical sponges with a single radio-opaque marker are readily visible on plain radiographs but occasionally the marker may be distorted by folding, or twisting over a period of time. Surgical sponges with radio-opaque markings on both sides or with multiple markings according to one embodiment of the present invention might enhance reliability of diagnosis in radiograph detection. Alternatively, the use of an embedded radio-chip in the sponge may be resorted to.

In a further variation, the characters representing the hemostatic content strength may be imprinted using color coding additionally, so that the users of the treated sponge will be doubly sure of the hemostatic strength.

### BRIEF DESCRIPTION OF THE DRAWING

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured. In the accompanying drawing:

FIG. 1 illustrates a hemostatic sponge in the form of a gauze which is pretreated with a predetermined amount of a selected hemostatic agent, the gauze being marked as containing 20% strength of the hemostatic material, for example;

FIG. 2 illustrates a hemostatic sponge similar to that in FIG. 1, but additionally including a radio-opaque element or marker;

FIG. 3 illustrates a hemostatic sponge similar to that in FIG. 1, but containing a plurality of markings of 20%; and,

FIG. 4 illustrates a hemostatic sponge similar to that in FIG. 1 but including the markings 20% on the front and back of the sponge.

### DETAILED DESCRIPTION

A detailed description of one or more embodiments of the invention is provided below along with accompanying figures that illustrate by way of example the principles of the invention. While the invention is described in connection with such embodiments, it should be understood that the invention is not limited to any embodiment. On the contrary, the scope of the invention is limited by the appended claims, and the invention encompasses numerous alternatives, modifications and equivalents. For the purpose of example, numerous specific details are set forth in the following description in order to provide a thorough understanding of the present invention.

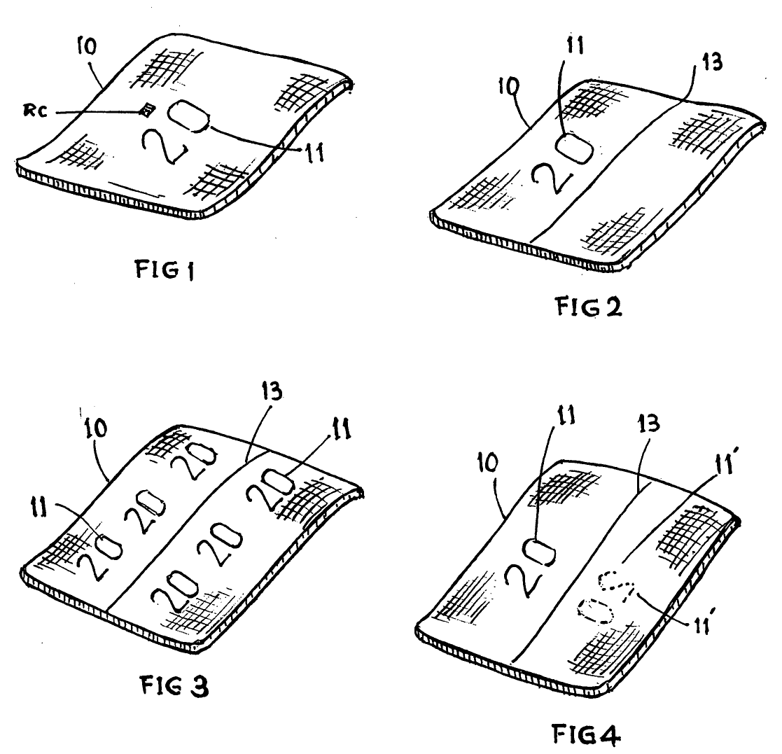

FIG. 1 shows a hemostatic sponge of the required material, dimension and the required ply. The material may be 100% cotton, for example USP type VII gauze, in sterile form, and in any suitable size as desired. Typical sizes as known to the users would include 2″×2″ 4 Ply or 8 Ply, 4″×4″ 8 Ply or 12 Ply, 4″×8″ 12 Ply, for example. Other sizes and number of plies are also known to those skilled in the art. The hemostatic sponges 10 are pretreated with known types of chosen hemostatic materials in liquid or powder form, and marked as shown at 11, to indicate the hemostatic content in the pretreated sponge. The hemostatic materials or agents may include one or more of Thrombin, Gelform, fibrinogen, cellulose, Aminocaproic acid, Tranexamic acid, Aprotinin®, Desmopressin® and ferric sulfate, and the visible indication of said predetermined quantity of the one or more hemostatic agents may comprise predetermined indications/markings on the sponge. In one form the markings may be matched with predetermined quantity/strength of one or more hemostatic agents corresponding to a user-chart. The user chart may be displayed on the sponge-package or in any other convenient/desired manner for easy use and visibility to the user.

In the example shown, the marking indicates 20% strength, which shows the measure of the expected efficacy of the pretreated sponge. With increased quantities of the hemostatic agent or material in the sponge of the same size and ply count, the sponges could be marked to indicate 40% or 60% or 80% or 100% as the case may be, to indicate the hemostatic agent content in the respective sponge. The surgeon or the user would deploy the required strength of the pretreated sponge depending on the need and the degree of bleeding noticed in a particular patient. In the absence of the pretreated and marked sponges of the present invention, the surgeon or the user would be applying the required hemostatic agent in unknown and unmeasured quantities on a sponge and applying the sponge with unknown amounts of the hemostatic agent to the surgical area. With the pretreated and marked sponges of the present invention, the surgeon or the user would simply seek the required strength and size of the hemostatic sponge and apply it to the bleeding area, with the knowledge of the properties of the pretreated gauze that is being applied. Also, the surgeon would be able to decide easily quickly if more hemostatic agent is needed or less, as the surgery progresses, depending on the patient response and the change in the degree of bleeding/clotting. By the use of the present teachings, the surgeon would simply switch to a different strength of the hemostatic sponge as guided by the markings thereon, in lieu of guessing the change in the requirements regarding the amount of hemostatic agent needed, as the surgery progresses.

FIG. 2 shows a pretreated sponge 10 similar to the one illustrated in FIG. 1 having the hemostatic agent content marking of 20% as shown at 11, but including a radio-opaque element shown by the numeral 13. The radio-opaque element 13 may be a thread or a filament or a strip, or even a chip (RC in FIG. 1) transmitting a signal to an external receiver. As known, the radio-opaque element would enable detection of any sponge that is inadvertently left in a patient and was not discovered earlier even after the usual counting routine at the operating table.

FIG. 3 illustrates a pretreated sponge 10 which has multiple markings 11 of the hemostatic content strength for easy visibility to the surgeon after the sponge has already been deployed. It is conceivable that the sparse markings on a sponge after insertion into a crevice are less visible, and multiple markings might alleviate the obscuring of a single marking. Consequently, in FIG. 3, the pretreated sponge includes a plurality of markings 11. It is noted however that the markings may be placed on both sides of the sponge, for easy visibility after the sponge has been deployed. The illustration in FIG. 3 shows a radio-opaque element 13 as in the FIG. 2 embodiment.

FIG. 4 illustrates a pretreated sponge 10 having the hemostatic agent content markings on both sides of the sponge, with the markings on the two sides being represented by 11 and 11′, so that the marking might be visible regardless of which way the sponge is facing, when it is handled or deployed. Also shown in the sponge of FIG. 4 is a radio-opaque element 13.

In another embodiment of the sponge of the present invention, the markings denoting the hemostatic agent content, e.g., 20%, 40%, 60% or 80% as the case may be constituted by color coding which is identified by a suitable indication on the packing of the sponges, or in the form of a user-chart/table readily available to the user. The color coding at a glance or the percentage marking would readily indicate to the surgeon regarding the strength of the hemostatic content in the pretreated sponge or article. The markings may alternatively be in the form of numeral markings such as “1 out of full strength of 5”, for example. The markings may be adapted to specific user/country needs if so desired. The color coding and the markings would assist in separately identifying the pretreated sponges/articles during manufacture, shipping, sale, storage and deployment so that the user would be able to select the required strength of the hemostatic agent content in the pretreated sponge/article. It is understood that that the embodiment of FIG. 4 might be designed to have color coding in addition to the specific marking of 20%, if the user so chooses. The predetermined markings on the sponge, said markings being matched with said predetermined quantity/strength of one or more hemostatic agents corresponding to a user-chart/table which is imprinted on the sponge/article package. The markings as aforesaid may be radio-opaque. Alternatively, the sponge/article may have at least one radio-opaque insert or an embedded radio-chip (RC in FIG. 1). The hemostatic sponge/article may be in a plurality of sizes and configurations as desired and convenient.

It is noted that in all the different embodiments, the pretreated sponges would be subjected to sterilization during manufacture in a known manner, so that they would be suitable for surgical use as necessary. Variations in the method of pretreatment and the desired control of the content of the hemostatic agent in the pretreated and marked sponges, as well as variations in the method of marking/identifying are conceivable and are within the ambit of the present invention. Hemostatic agent herein is to be understood as a substance that assists in causing hemostasis and may be in powder or fluid form, and may be chosen from a group including Thrombin, Gelform, Fibrinogen, or other hemostatic substances.

The features and advantages of the pretreated marked hemostatic sponges described above include the following:

### 1. The surgeon or the user would not have to lose time in manually applying the required hemostatic agent on the surgical sponge since it is pretreated.

2. The hemostatic content in the pretreated sponge is controlled at application (during manufacture) and identified by either numerical markings on the sponge or color coding thereof, so that the user would beforehand know the expected efficacy of the rating of a specifically marked sponge.

### 3. Since some embodiments include the radio-opaque or radio-chip elements, the sponges retain the advantages offered by the radio-opaque or the radio-chip elements in an eventuality.

4. Multiple markings of the numerals on the pretreated sponges to indicate the hemostatic agent content enhance visibility and easy identification of the numeral markings even when the sponges become twisted or deformed after placement in-situ during surgery.

### 5. In one embodiment, since the numerals marked on the sponge to indicate the hemostatic agent content can be made radio-opaque, there may be cost-saving in not having to provide an additional separate radio-opaque insert.

### 6. The reliability and convenience-factor which the user or surgeon realizes in deploying the pretreated marked sponges as taught hereinabove are significant.

In the foregoing detailed description of embodiments of the invention, various features are grouped together in a single exemplary embodiment for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims are hereby incorporated into the detailed description of embodiments of the invention, with each claim standing on its own as a separate embodiment. It is understood that the above description is intended to be illustrative, and not restrictive. It is intended to cover all alternatives, modifications and equivalents as may be included within the spirit and scope of the invention as defined in the appended claims. Many other embodiments will be apparent to those of skill in the art upon reviewing the above description. The scope of the invention should therefore be determined with reference to the appended claims, along with the full scope of equivalents to which such claims are entitled. In the above text and the appended claims, the terms “comprising” and “wherein,” where used, are to be understood as plain-English equivalents of the terms “including” and “in which” respectively.

1. A hemostatic article which is pretreated with a predetermined quantity of one or more hemostatic agents, the hemostatic article including a visible indication thereon, of said predetermined quantity of said one or more hemostatic agents.

2. The hemostatic article as in claim 1, wherein said hemostatic agents are in powder form, and wherein said visible indication comprises percentage quantity markings such as 20%, 40%, 60%, 80% on each said hemostatic article.

3. The hemostatic article as in claim 2, wherein said article comprises a sponge and includes at least one radio-opaque element included therein.

4. The hemostatic article as in claim 3, wherein said radio-opaque element comprises a radio-opaque thread.

5. The hemostatic article as in claim 2, wherein said article comprises a sponge, and wherein said percentage quantity markings are printed to be boldly visible on said hemostatic article, said markings being radio-opaque.

6. The hemostatic article as in claim 2, wherein said article comprises a sponge, and wherein said markings are provided on two sides of the sponge.

7. The hemostatic article as in claim 2, wherein said article comprises gauze, and said percentage quantity markings are provided on two sides of the gauze.

8. The hemostatic article as in claim 2, wherein said article comprises gauze, and said percentage quantity markings are in the form of multiple markings on each side of the gauze.

9. The hemostatic article as in claim 1, wherein said visible indication comprises color coding using different colors to represent said predetermined quantity of said one or more hemostatic agents as percentages including 20%, 40%, 60% and 80%.

10. The hemostatic article as in claim 5, wherein said percentage quantity markings are provided on two sides of the sponge.

11. A hemostatic sponge including gauze material which is pretreated with a predetermined quantity of one or more hemostatic agents, the hemostatic sponge including a visible indication of said predetermined quantity of said one or more hemostatic agents.

12. A hemostatic sponge including gauze material which is pretreated with a predetermined quantity of one or more hemostatic agents, the hemostatic sponge including a visible indication of said predetermined quantity of said one or more hemostatic agents, said visible indication comprising markings on each said hemostatic article to indicate a percentage content of said predetermined quantity of one or more hemostatic agents.

13. The hemostatic sponge as in claim 12, wherein said hemostatic agents include one or more of Thrombin, Gelform, fibrinogen, cellulose, Aminocaproic acid, Tranexamic acid, Aprotinin®, Desmopressin® and ferric sulfate, and wherein said markings include percentages such as 20%, 40%, 60% and, 80%.

14. The hemostatic sponge including gauze material as in claim 13, wherein said one or more hemostatic agents comprise materials in powder form.

15. The hemostatic sponge as in claim 11, wherein said hemostatic agents include one or more of Thrombin, Gelform, fibrinogen, cellulose, Aminocaproic acid, Tranexamic acid, Aprotinin®, Desmopressin® and ferric sulfate, and wherein said visible indication of said predetermined quantity of said one or more hemostatic agents comprises predetermined markings on the sponge, said markings being matched with said predetermined quantity/strength of one or more hemostatic agents corresponding to a user-chart.

16. The hemostatic sponge as in claim 15, wherein said predetermined markings comprise percentage content.

17. The hemostatic sponge as in claim 15, wherein said predetermined markings comprise numerals, and said sponge includes an embedded radio-chip.

18. The hemostatic sponge as in claim 15, wherein said markings are radio-opaque.

19. The hemostatic sponge as in claim 15, wherein said hemostatic sponge is in a plurality of sizes and configurations.

20. The hemostatic sponge as in claim 11, wherein the gauze material includes at least one radio-opaque element included therein.
